(19) United States
(12) Patent Application Publication
(10) Pub. No.: US 2012/0065648 A1
(43) Pub. Date: Mar. 15, 2012

(54) SUTURE CLOSURE DEVICE

(75) Inventor: Wouter E. Roorda, Palo Alto, CA (US)

(73) Assignee: ABBOTT CARDIOVASCULAR SYSTEMS, INC., Santa Clara, CA (US)

(21) Appl. No.: 12/879,636

(22) Filed: Sep. 10, 2010

(51) Int. Cl. A61B 17/04

(52) U.S. Cl. 606/148

(57) ABSTRACT

A closure device is provided for clamping sutures when closing an opening in tissue. The closure device includes an inner part and an outer part. Sutures drawn through the inner part and the outer part are clamped and held in place when the inner part is pressed together with the outer part.

### BACKGROUND OF THE INVENTION

1. The Field of the Invention

Embodiments of the invention relate generally to medical devices. More particularly, embodiments of the invention relate to medical devices for closing holes in tissue.

2. The Relevant Technology

Catheterization and interventional procedures, such as angioplasty or stenting, are generally performed by inserting a hollow needle through a patient's skin and tissue into the patient's vascular system. A guide wire may be advanced through the needle and into the patient's blood vessel accessed by the needle. The needle is then removed, enabling an introducer sheath to be advanced over the guide wire into the vessel, e.g., in conjunction with or subsequent to a dilator.

A catheter or other device may then be advanced through a lumen of the introducer sheath and over the guide wire into a position for performing a medical procedure. Thus, the introducer sheath may facilitate introducing various devices into the vessel, while minimizing trauma to the vessel wall and/or minimizing blood loss during a procedure.

Upon completing the procedure, the devices and introducer sheath are removed, leaving a puncture site in the vessel wall. Traditionally, external pressure would be applied to the puncture site until clotting and wound sealing occur; however, the patient must remain bedridden for a substantial period after clotting to ensure closure of the wound. This procedure, however, may be time consuming and expensive, requiring as much as an hour of a physician's or nurse's time. It is also uncomfortable for the patient and requires that the patient remain immobilized in the operating room, catheter lab, or holding area. In addition, a risk of hematoma exists from bleeding before hemostasis occurs.

### BRIEF SUMMARY OF THE INVENTION

An embodiment of a closure device, also referred to herein as a suture clamp, is provided to close openings in tissue or to aid in closing openings in tissue. In one embodiment, the closure device for clamping sutures includes an inner part having an opening that extends from a distal end to a proximal end. The closure device also includes an outer part that defines an aperture. The inner part is received into the aperture of the outer part to connect the closure device and to clamp sutures that have been drawn through the opening in the inner part. Insertion of the inner part into the outer part closes the opening on the sutures.

In another embodiment, the closure device includes an inner part having an opening formed therein. An engagement mechanism is formed on an outer surface or wall of the inner part. The closure device may also include an outer part. A corresponding engagement mechanism is formed on an inner surface of the outer part. The engagement mechanisms engage to connect the inner part with the outer part.

Embodiments are also directed to methods for closing an opening is tissue. After placing sutures around the opening in the tissue, the sutures are drawn through an inner part and an outer part of a closure device. The sutures are then tightened to close the opening in the tissue. Finally, the inner part is pressed together with the outer part to clamp the sutures in the closure device and hold the opening in the tissue closed. Clamping the sutures holds the sutures in place and prevents the sutures from loosening.

These and other advantages and features of the present invention will become more fully apparent from the following description and appended claims, or may be learned by the practice of the invention as set forth hereinafter.

### BRIEF DESCRIPTION OF THE DRAWINGS

To further clarify at least some of the advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 shows an illustrative example of a closure device for closing an opening in tissue;

FIG. 2A illustrates an expanded perspective view of a closure device including sutures that are drawn through an outer part and an inner part of the closure device;

FIG. 2B illustrates a perspective view of the closure device after the inner part and the outer part have been connected and the sutures are clamped in an opening formed in the inner part;

FIG. 2C illustrates another embodiment of a closure device for closing the opening in tissue where the sutures are clamped between the inner part and the outer part;

FIG. 3A shows an illustrative perspective view of a closure device that includes projections formed on the inner part and on the outer part to connect the inner part with the outer part;

FIG. 3B illustrates how the projections engage to connect the closure device;

FIG. 4 shows an alternative embodiment of a closure device;

FIG. 5 shows an example of the closure device of FIG. 1 after the closure device is deployed to close an opening in a vessel;

FIG. 6 shows an illustrative example of a closure device for securing sutures;

FIG. 7 shows an illustrative example of a closure device for clamping sutures; and

FIG. 8 shows an illustrative example of delivery device for deploying a closure device.

### DETAILED DESCRIPTION OF THE PREFERRED EMBODIMENTS

Devices and methods are disclosed herein for managing access through tissue and in particular to suture mediated closure devices. Several examples are described below in which a closure device may be deployed to close an opening in tissue or to aid in closing the opening in the tissue. Some embodiments may be used to close and/or substantially close openings in a body lumen, such as blood vessel, or other tissue formed during a diagnostic, therapeutic, and/or other procedure. Embodiments of the closure device or closure elements included therein may be made of any suitable material, including a bioabsorbable, bioresobable or biodegradable material.

Embodiments of the invention relate to suture mediated closure devices and include a closure device configured to aid in closing an opening in tissue, such as a vessel, that provides access to a body lumen. In one embodiment, the closure device includes an inner part and an outer part. The outer part accommodates the inner part to hold the sutures in place and keep the opening closed. After placing sutures as needed to draw the opening closed, the sutures can be drawn into the inner part and into the outer part or between the inner and outer part of the closure device. The inner part and the outer part are then connected to clamp or cinch the sutures to maintain the sutures in place and/or to keep the sutured opening substantially closed. The inner part and the outer part can cooperate to clamp sutures, for example using compression. In this example, the inner part compressively fits with the outer part to hold the sutures and keep the sutured opening substantially closed.

The closure of a large hole or opening in tissue, such as an arteriotomy, often requires a couple of steps to close. The first step includes the placement of sutures in the tissue around or proximate the opening. Sutures are placed, for instance, in walls of the tissue surrounding the opening providing access to a body lumen. After the sutures are placed, the sutures are cinched or tightened and then locked or held in place with a closure device. By locking or holding the sutures in place, the tightened sutures remain in place and/or the sutured opening remains closed.

The following discussion references an opening formed in a vessel, which is an example of tissue that may be sutured. One of skill in the art, with the benefit of the present disclosure, can appreciate that embodiments of the invention can be used to close openings in tissue, including openings in tissue that provide access to body lumens or that are used to perform medical procedures.

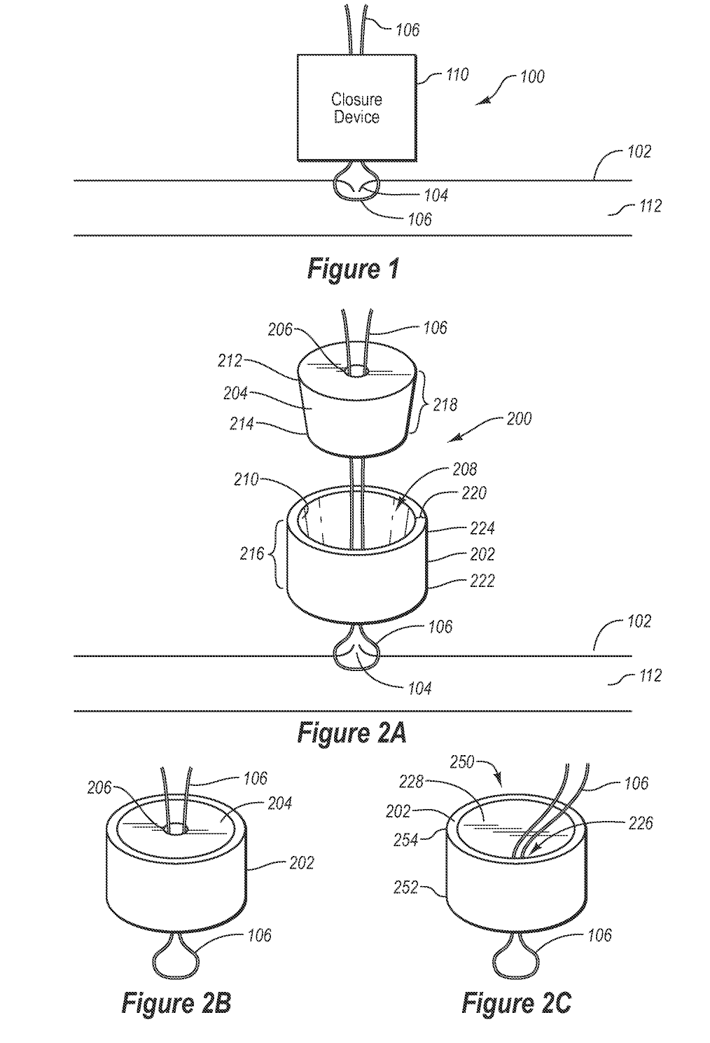

FIG. 1 shows an illustrative embodiment of a system 100 for closing an opening 104 in a vessel 102. In this example, the opening 104 may be an arteriotomy or formed during another procedure. After the procedure is finished and/or the medical devices used during the procedure have been withdrawn from the lumen 112, the opening 104 needs to be closed.

In this example, sutures 106 are placed in the wall of the vessel 102 around the opening 104. The sutures 106 are then drawn through a closure device 110 and tightened to close or substantially close the opening 104. Tightening the sutures 106 may pull the walls of the vessel 102 proximate the opening 104 together to close the opening 104. The closure device 110 can hold the sutures 106 tightly in place such that the opening 104 remains closed or substantially closed while healing.

The closure device 110 can be configured to engage the sutures 106 without requiring a knot in the sutures 106. The closure device 110 can clamp the sutures 106, for example using compression, to hold the sutures 106 in place.

FIG. 2A shows an illustrative embodiment of a closure device 200, which is an example of the closure device 110. The closure device 200 includes an outer part 202 and an inner part 204. The inner part 204 includes an opening 206 formed in the body of the inner part 204. The opening 206 is typically formed in the center of the inner part 204 and extends from a proximal end 212 to a distal end 214. The opening 206, however, may be non-centrically placed or located in the inner part 204. In some embodiment, the opening 206 may not be present and the sutures 106 are clamped between an outer wall 218 of the inner part 204 and an inner wall 210 of the outer part 202.

After sutures 106 are placed in the walls of the vessel 102, the sutures 106 are drawn through the outer part 202 and through the opening 206 of the inner part 204. The outer part 202 can be a hollow tube or ring like structure in one embodiment, although the shape can vary, and the inner part 204 can be a plug that fits in the outer part 202. For example, the outer part 202 may be frusto-conical or otherwise shaped. In addition, the outer part 202 may include projections on at least one surface.

FIG. 2B shows an illustrative embodiment of the closure device 200 after the inner part 204 and the outer part 202 have been connected. With reference to FIGS. 2A and 2B, the inner part 204 is received into an aperture 208 of the outer part 202. As the inner part 204 is inserted into the aperture 208 of the outer part 202, the inner part 202 may experience some compression. The compression experienced by the inner part 204 may at least partially close the opening 206 to clamp and hold the sutures 106. The compression is sufficient to hold the sutures 106 in place such that the opening 104 may be held closed. The compression may be applied to the sutures 106 by the outer part 202, the inner part 204, or a combination thereof.

The outer part 202 includes a body defining the aperture 208. The outer part 202 includes an inner surface or wall 210 that substantially corresponds to an outer surface or wall 212 of the inner part 204. When the inner part 204 and the outer part 202 are connected, the outer wall 212 and the inner wall 210 are typically at least partially in contact. In one example, the outer part 202, via the inner wall 210, may exert a compressive force on the inner part 204. In one example, the outer part 202 may be more rigid than the inner part 204 such that the inner part 204 compresses when the inner part 202 and the outer part 204 are connected. Thus, the material composition of the outer part 202 and the inner part 204 may be different to facilitate clamping the sutures 106 in the opening 206. The inner part 204 may be more compressible such that it compresses when placed in the aperture 208 of the outer part 202.

The outer part 202 is shown in FIGS. 2A-2B as being circular or ring-like. However, the outer part 202 may have another configuration or shape. For example, the outer part 202 may be circular, elliptical, square, or any other suitable shape. The shape of the inner part 204 may substantially match the shape of the outer part 202. In this example, the inner part 204 is also circular or ring like to match the shape of the outer part 202. In one embodiment, the inner part 204 and the outer part 202 are shaped such that the inner part 204 plugs the outer part 202 in a manner that holds the sutures 106. In this example, the inner part 204 plugs into the outer part 202 to close the opening 206 and clamp the sutures 106.

In one example, the inner part 204 may be slightly larger than the aperture 208. More specifically, a cross sectional area of the inner part 204 bounded by the outer wall 218 may be slightly larger than a cross sectional area of the outer part 202 bounded by the inner wall 210. This may ensure that the inner part 204 compresses when connected with the outer part 202 to hold the sutures 106.

In addition, at least a portion of the inner part 204 may be tapered between the distal end 214 and the proximal end 212 to facilitate insertion of the inner part 204 into the outer part 202. The aperture 208 may have a reverse taper to accommodate the taper of the inner part 204. The taper ensures that the distal end 214 of the inner part 204 can be easily introduced into a proximal end of the outer part 202. In fact, the distal end 214 may not engage the inner wall 210 until the inner part 204 is partially inserted into the aperture 208. Because the inner part 204 may be slightly larger than the aperture 208, the outer part 202 compresses the inner part 204 to reduce the size of the opening 206 about the sutures 106 and hold the sutures 106 in place when the connection between the inner part 204 and the outer part 202 is complete.

The dimensions of the outer part 202 and the inner part 204 may vary according to the size of the opening 104 in the vessel 102. In addition, the inner part 204 may include multiple openings 206 such that multiple sutures 106 may be clamped and/or the sutures 106 can be clamped in multiple locations if necessary. A height 216 of the outer part 202 may be between 0.5 and 5 mm or between 1 and 3 mm or other range. A width of a body 220 may be between 1 and 10 mm or between 2 and 4 mm. As previously stated, the width may vary when the body 220 is tapered. Thus the width of the body 220 may thicken along the length moving from the proximal end to the distal end. Similarly, a width of the inner part 204 may vary according to a taper. The taper of the inner part 204 may also mirror the taper of the body 220. A height of the inner part 204 may substantially match the height 216 such that the inner part 204 is flush with the outer part 202 when connected. Alternatively, the inner part 202 may extend on either a distal end 222 and/or a proximal end 224 of the outer part 202. One of skill in the art can appreciate that the dimensions are provided by way of example only and not limitation.

During operation, the sutures 106 are placed in the walls of the vessel 102 surrounding the opening 104 after completion of a procedure when the opening 104 is being prepared for closure. The sutures 106 are then drawn through the aperture 208 of the outer part 202 and through the opening 206 of the inner part 204. After pulling the sutures 106 tight to close the opening 104, the inner part 204 can be pressed into the outer part 202. Pressing the inner part 204 into the outer part 202 closes the opening 206 to clamp the sutures 106 in place and limit and/or prevent the sutures 106 from loosening. The inner part 204 and the outer part 202 are configured such that pressing the inner part 204 and the outer part 202 together at least partially closes the opening 206 about the sutures 106 to hold the sutures 106 in place. Thus, the sutures 106 are held within the at least partially closed opening 206. The inner part 204 may remain fixed with respect to the other part 202 due to various forces, such as friction, compression, other forces or combination thereof. Thus, the inner part 204 and the outer part 202 remain connected once the inner part 204 is pressed into or inserted into the outer part 202.

FIG. 2C illustrates another example of a closure device 250, which is an example of the closure device 110. In this example, an inner part 228 is shown without an opening in the body. The sutures 106 are clamped between the inner part 228 and the outer part 202, rather than within an opening in the inner part of 228. Pressing the inner part 228 and the outer part 202 together clamps the sutures 106 when the sutures 106 are placed between the inner part 228 and the outer part 202. FIG. 2C shows the sutures 106 clamped at a location 226 between the inner part 228 and the outer part 202. In this example, the inner part 228 does not include the opening 206 like the inner part 204, although the outer part 202 may be the same as shown in FIGS. 2A and 2B. In other examples, at least one suture may be clamped both within an opening and between the two parts. Because the outer wall of the inner part 228 and the inner wall of the outer part 202 substantially match, the inner part 228 and the outer part 202 press against the sutures 106 between a distal end 252 of the closure device 250 and a proximal end 254 of the closure device 250. In this example, the sutures 106 are clamped along the height of the closure device 250 from the distal end 252 to the proximal end 254 and exit the closure device 250 at the location 226.

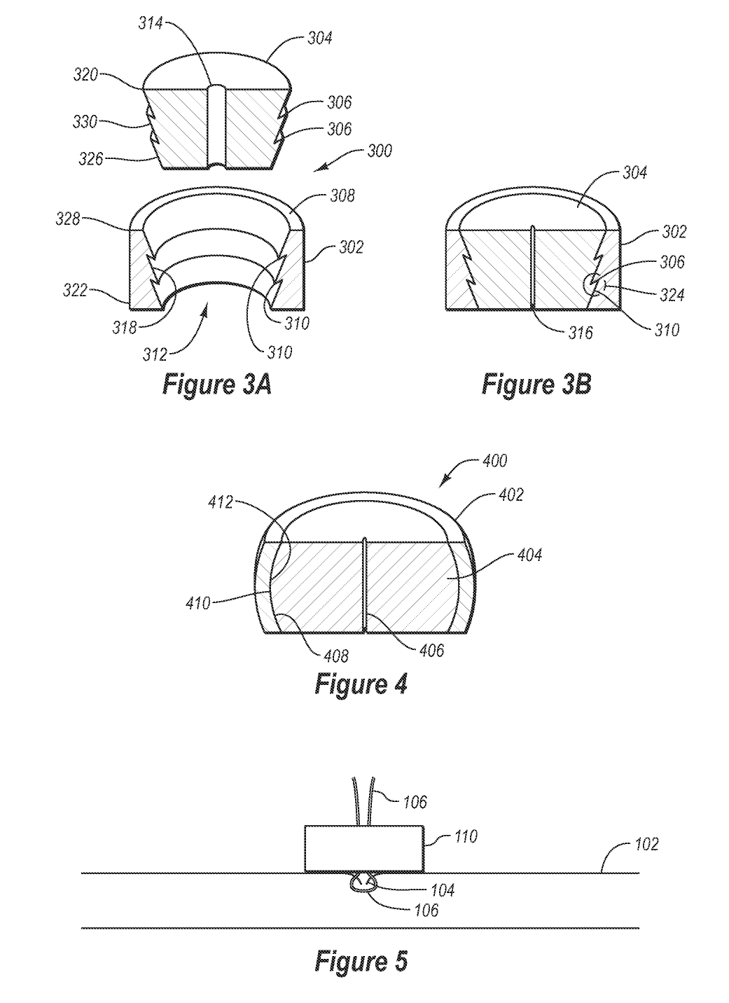

FIGS. 3A and 3B show an illustrative embodiment of a closure device 300, which is an example of the closure device 110. FIG. 3A shows the closure device 300 prior to deployment and FIG. 3B shows the closure device 300 after deployment. The closure device 300 includes an outer part 302 and an inner part 304 that cooperate to clamp sutures. The inner part 304 includes projections 306, which are an example of an engagement mechanism. The projections 306 extend out from a body of the inner part 304 and away from an opening 314 in this example. The projections 306 are arranged on an outer surface or wall 330. The projections 306 may be disposed at least partially around the wall 330. The projections 306 may include one or more projections 306 that are spaced on the wall 330. Projections 310 are formed on an inner wall of the outer part 302.

The projections 310 are typically configured to engage the projections 306. In one example, however, projections may only be formed on the inner part 304 or on the outer part 304. The friction provided by a single instance of the projections is sufficient to keep the inner part 304 and the outer part 302 connected. In this example, the engagement mechanism on one of the parts includes projections while the engagement mechanism on the other one of the parts is the wall of the part.

FIG. 3A, however, illustrates two projections: the projection 306 arranged on the inner part 304 and a projection 310 arranged on the outer part 302 the distal end 326. The projections 306, 310 are typically located in corresponding locations on the outer wall 330 of the inner part 304 and an inner wall 318 of the outer part 302, respectively.

The outer part 302 includes projections 310 that extend out from a wall 308. The projections 310 are an example of a complementary engagement mechanism that cooperate with the projections 306 on the inner part 304 to keep the inner part 304 and the outer part 302 from being separated after being connected. The projections 310 extend into an aperture 312 of the outer part 302. The projections 306 and the projections 310 are configured to permit the inner part 304 to be inserted into the aperture 312 while preventing the inner part 304 from being extracted or separated from the outer part 302 once the inner part 304 and the outer part 302 are pressed together. The projections 306 and 310 ensure that the closure device 300 functions to mediate closure of the opening by the sutures by keeping the inner part 304 connected with the outer part 302 such that the sutures remain clamped for as long as necessary. The closure devices disclosed herein can be easily removed, if necessary, by snipping the sutures between the opening in the vessel and the closure device.

The projections 306 and the projections 310 are oriented to permit insertion of the inner part 304 while resisting extraction or removal of the inner part 304 from the outer part 302. In this example, the projections 306 and 310 are oriented such that the projections 306 slip past the projections 310 during insertion of the inner part 304 into the outer part 302. However, the orientation of the projections 306 and 310 cause the projections to engage when movement of the inner part 302 in a proximal direction is attempted.

In this example, the projections 306 are generally directed in a proximal direction towards a proximal end 320. The projections 310 are generally oriented in a distal direction towards a distal end 322. The orientation of the projections 306 and 310 enable distal movement but resist proximal movement once the inner part 304 engages the outer part 302. FIG. 3B illustrates the engagement 324 between the projections 306 and 310 after the inner part 304 and the outer part 302 are connected. In addition, the outer part 302 may exert a compressive force on the inner part 304 to close the opening 314 when connected.

The inner wall or surface 318 of the outer part 302 may also be generally sloped or tapered. The surface 330 of the inner part 304 may also be generally tapered or sloped, although the surface 330 is tapered in the complementary direction such that the inner part 304 can fully fit in the outer part 302 as illustrated in FIG. 3B. Tapering the surfaces 318 and 330 may ensure that the inner part 304 cannot be pushed through the outer part 302 while the projections 306 and 310 cooperate to prevent the inner part 304 from coming loose.

As previously stated, the outer part 302 may be more rigid and less flexible than the inner part 304. The outer part 302 may be formed of metals or polymers, examples of which are stainless steel or titanium, resp. hard poly-urethanes, poly-acrylates, poly-amides, polyesters or poly-olefines. If desired, biodegradable materials like Mg-alloys, resp poly-lactates or poly-glycolides can be used.

The inner part 304 may be formed of elastically deformable polymers, like soft poly-urethanes, poly-acrylates and poly-esters, or plasticized versions of harder polymers. Biodegradable elastically deformable materials include poly-caprolactone.

Those skilled in the art will be able to identify many more materials, since the nature of the disclosed device allows for a wide range of materials to be used, basically constrained by requirements of biocompatibility, sterilizability and Young's modulus.

As previously stated, insertion of the inner part 304 into the outer part 302 at least partially closes the opening 314 shown in FIG. 3A, which is illustrated as closed in FIG. 3B. The at least partially closed opening 316 clamps the sutures that hold the opening in the vessel or other tissue at least partially closed. Thus, connection of the inner part 304 with the outer part 302 at least partially closes the opening 314 such that the sutures are clamped in the opening 316. Because the opening 314 extends between the proximal end 320 and the distal end 326, the sutures may be clamped along a relatively large surface area.

When the inner part 304 is tapered as previously discussed, the distal end 326 is smaller (e.g, in circumference or in cross sectional area) than the proximal end 320. In this case, the taper in the outer part 302 is formed such that a wall at a distal end 322 is thicker than the wall at the proximal end 328 of the outer part 304. Stated another way, the aperture 312 in the outer part 302 is narrower at the distal end 322.

The projections 306 and 310 may also be flexible. In this example, the projections 306 and 310 are oriented such that the projections can flex to permit insertion of the inner part 304. The orientation of the projections 306 and 310, however, prevents or resists extraction of the inner part 304 at the same time. The engagement mechanism between the inner part 304 and the outer part 302 can include other arrangements as well. For example, the projections may have relatively smooth contours or sharp contours. The projections can be oriented in other directions as well. The projections can be symmetrical as well. The projections may only be formed on one of the inner part 304 and the outer part 304.

In another example, the projections by be formed as threads such that the inner part 304 may rotate into the outer part 302. In this manner, the opening 314 can gradually close as the inner part 304 is tightened. Additionally, the inner part 304 or the outer part 302 may include projections without a corresponding projection to the other part. For example, the outer part 302 may include a projection on its proximal end 328 that may be sufficient to retain the inner part 304.

FIG. 4 illustrates another embodiment of a closure device 400, which is an example of the closure device 110. In FIG. 4, an outer wall 412 of an inner part 404 is curved (concave in this example) and an inner wall 410 of an outer part 402 is correspondingly curved (convex in this example). The outer part 402 may be flexible and expandable such that it can adapt to the outer wall 412 of the inner part 404. In addition, the outer part 402 may be configured such that it exerts a compressive force on the inner part 404 at least partially to close an opening 406 and clamp the sutures in the inner part 404.

An interface between the inner part 404 and the outer part 402 is shaped to limit or prevent the outer part 402 from becoming displaced from the inner part 404 while the opening in the tissue heals. In this example, the aperture (shown as 312 in FIG. 3A for the outer part 302) in the outer part 402 may be smaller dimensionally than the inner part 404. This ensures that when the outer part 402 is placed around the outer wall 412, the outer part 402 exerts a compressive force directed towards the opening 406 in order to close the opening 406 on the sutures.

FIG. 5 shows an illustrative example of the closure device 110 after deployment. During operation or use of the closure device 110, sutures are placed in the walls of the vessel (or other tissue) surrounding the opening. The sutures are then fed through the aperture of the outer part and/or through the opening in the inner part. The sutures can be drawn through the opening in the inner part using a snare device if necessary. After the sutures are tightened, the inner part is pressed into or joined with the outer part. Preferably, the closure device 110 is adjacent the opening 104 and may be touching or in contact with the vessel 102 in some embodiments. Close placement of the closure device 110 to the opening 104 may ensure that the clamped sutures keep the opening 104 at least partially closed for healing. In this example, the opening in the closure device 110 will typically be centrally located above the opening 104. Of course this may depend on how the sutures are placed. The closure device 110 can be removed by snipping at the sutures between the vessel 102 and the closure device 110. Alternatively, the closure device 110 and/or the sutures 106 may dissolve or be absorbed.

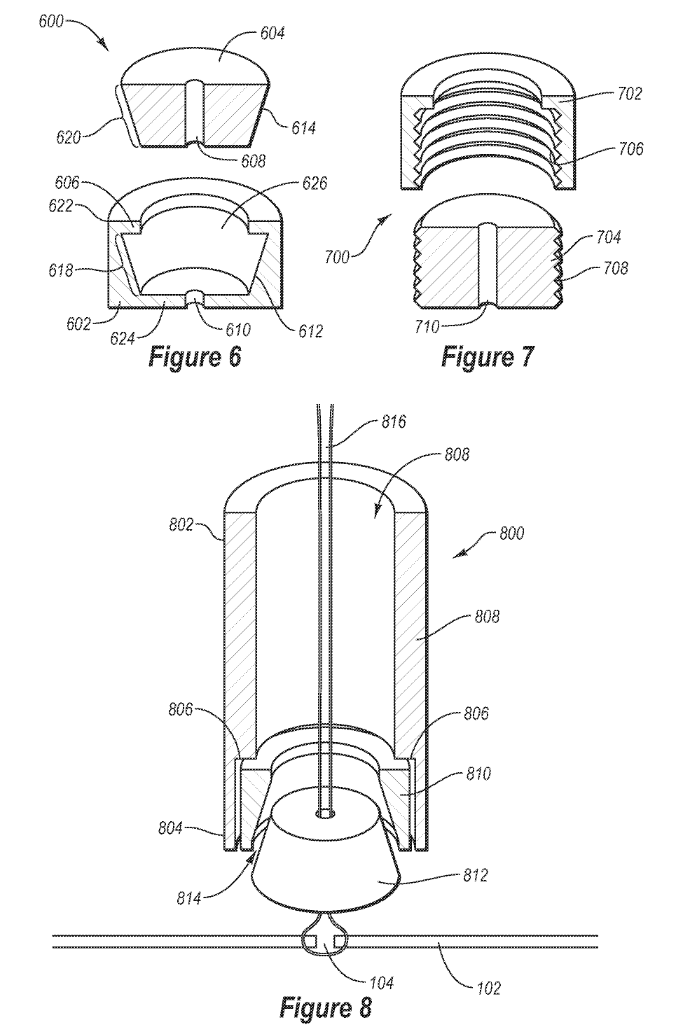

FIG. 6 shows an illustrative example of a closure device 600. The closure device 600 is an example of the closure device 110. The closure device 600 includes an outer part 602 and an inner part 604. Sutures are pulled through an opening 608 and through an aperture 610 as previously described. When the inner part 604 is inserted into the outer part 602, the device 600 clamps sutures in the opening 608.

More specifically, the outer part 602 includes an inner surface 618 that corresponds with an outer surface 620. The outer part 602 When connected, the inner surface 618 exerts a force on the inner part 604 to close the opening 608 on the sutures. In this example, the inner surface 618 includes a taper 612 that matches a taper 614.

The outer part 602 includes a securing projection 606. When the inner part 604 is inserted into the outer part 602, an end 622 of the outer part 602 expands to accommodate entry of the inner part 604. Once the inner part 604 is fully inserted into the outer part 602, the projection 606 snaps back and secures the inner part 604 within the outer part 602. The projection 606 prevents the inner part 604 from becoming separated from the outer part 602.

FIG. 6 further illustrates that the outer part 602 may include a base 624, although the base 624 may also be formed as a projection such that the inner part 604 is secured between a proximal and a distal projection. The base 624 provides a stop for insertion of the inner part 604. The outer part 602 defines a cavity 626 that is shaped to accommodate the inner part 604. The cavity 626 may be slightly smaller than the inner part 604 to insure that the opening 608 clamps the sutures when the inner part 604 is connected with the outer part 602.

FIG. 7 shows an illustrative example of a closure device 700. The closure device 700 is an example of the closure device 110. The closure device 700 includes an inner part 704 and an inner part 702. The closure device 700, similar to other embodiments discloses herein, clamps sutures in an opening 710. In this example, the inner part 704 includes threads 708 on an outer surface that are configured to connect with threads 706 on an inner surface of the outer part 702. As the inner part 704 is rotated or screwed into the outer part 702, the opening 710 closes on the sutures.

FIG. 8 illustrates an example of a delivery device 800 for deploying a closure device. The delivery device 800 may deploy, for example, embodiments of the closure device 110. The delivery device 800 includes a shaft 808 having a distal end 804 and a proximal end 802. The distal end 804 includes a recess 806 configured to receive an outer part 810 of a closure device 814, which is an example of the closure device 110. The outer part 810 may be held in place in the recess 806 by friction in this example, although the distal end 804 may include small tabs to aid in retaining the outer part 810.

In this example, the sutures 106 are drawn through the inner part 812 as previously described. The sutures 106 are then drawn through the outer part 810 and through an opening 808 in the delivery device 800. During deployment of the closure device 800, a user can grasp an end 816 of the sutures 106 and pull proximally while pushing the shaft 808 distally. This ensures that the inner part 812 is positioned adjacent the wall of the vessel 102 when the outer part 810 is pushed or urged to connect with the inner part 812.

Thus, the delivery device 800 is used to connect the inner part 812 with the outer part 810 as described herein in various embodiments while positioning the closure device 814 in the appropriate location as the sutures 106 are clamped in the closure device 814.

FIG. 8 also illustrates that the orientation of the closure device 814 can vary. From the perspective of a user or of the delivery device, the terms distal and proximal may depend on the orientation of the closure device 814. In FIG. 8, the sutures 106 are drawn through the inner part 812 first. In other examples, the sutures 106 may be drawn through the outer part first as disclosed herein. Other embodiments of the closure device disclosed herein can be deployed in different orientations. Depending on the orientation, the sutures may be drawn through the inner part before being drawn through the outer part, as illustrated in FIG. 8. Alternatively, the sutures may be drawn through the outer part before being drawn through the inner part.

The closure device or portions thereof can be comprised of a variety of known suitable materials (which may be deformable), including stainless steel, silver, platinum, tantalum, palladium, nickel, titanium, nitinol, nitinol having tertiary materials, niobium-tantalum alloy optionally doped with a tertiary material cobalt-chromium alloys, or other known biocompatible materials. Such biocompatible materials can include a suitable biocompatible polymer in addition to or in place of a suitable metal. A device or member can include biodegradable or bioabsorbable materials, which can be either plastically deformable or capable of being set in the deployed configuration.

In one embodiment, the closure device is made from a superelastic alloy such as nickel-titanium or nitinol, and includes a ternary element selected from the group of chemical elements consisting of iridium, platinum, gold, rhenium, tungsten, palladium, rhodium, tantalum, silver, ruthenium, or hafnium. The added ternary element improves the radiopacity of the nitinol closure device or other medical device comparable to that of a stainless steel device or member of the same size and shape coated with a thin layer of gold. The nitinol device or member may have improved radiopacity yet may retain its superelastic and shape memory behavior and further maintains a thin strut/wall thickness for high flexibility.

In one embodiment, the closure device or portions thereof can be made at least in part of a high strength, low modulus metal alloy comprising Niobium, Tantalum, and at least one element selected from the group consisting of Zirconium, Tungsten, and Molybdenum. The closure device according to the present invention may provide superior characteristics with regard to bio-compatibility, radio-opacity and MRI compatibility.

Furthermore, the closure device or portions thereof can be formed from a ceramic material. In one aspect, the ceramic can be a biocompatible ceramic that optionally can be porous. Examples of suitable ceramic materials include hydroxylapatite, mullite, crystalline oxides, non-crystalline oxides, carbides, nitrides, silicides, borides, phosphides, sulfides, tellurides, selenides, aluminum oxide, silicon oxide, titanium oxide, zirconium oxide, alumina-zirconia, silicon carbide, titanium carbide, titanium boride, aluminum nitride, silicon nitride, ferrites, iron sulfide, and the like. Optionally, the ceramic can be provided as sinterable particles that are sintered into the shape of a closure device or layer thereof.

Moreover, the closure device body or portions thereof can include a radiopaque material to increase visibility during placement. Optionally, the radiopaque material can be a layer or coating any portion of the device or member. The radiopaque materials can be platinum, tungsten, silver, stainless steel, gold, tantalum, bismuth, barium sulfate, or a similar material.

It is further contemplated that the external surface and/or internal surface of the devices or members (e.g., exterior and luminal surfaces) as well as the entire body can be coated with another material having a composition different from the primary material. The use of a different material to coat the surfaces can be beneficial for imparting additional properties to the device or member, such as providing radiopaque characteristics, drug-reservoirs, and improved biocompatibility.

In one embodiment, at least one biocompatible polymeric layer can be a coating that is applied over the entire device or member, or to select portions. Examples of such biocompatible polymeric materials can include a suitable hydrogel, hydrophilic polymer, hydrophobic polymer biodegradable polymers, bioabsorbable polymers, and monomers thereof. Examples of such polymers can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, polyethylenes, polypropylenes, polyaliphatics, polyvinylalcohols, polyvinylacetates, hydrophobic/hydrophilic copolymers, alkylvinylalcohol copolymers, ethylenevinylalcohol copolymers (EVAL), propylenevinylalcohol copolymers, polyvinylpyrrolidone (PVP), combinations thereof, polymers having monomers thereof, or the like. Additionally, the coating can include hydrophilic and/or hydrophobic compounds, polypeptides, proteins, amino acids, polyethylene glycols, parylene, heparin, phosphorylcholine, or the like.

The coatings can also be provided on the device or member to facilitate the loading or delivery of beneficial agents or drugs, such as therapeutic agents, pharmaceuticals and radiation therapies. As such, the material and/or holes can be filled and/or coated with a biodegradable material.

In addition to various medical devices or members, the coatings on these devices or members may be used to deliver therapeutic and pharmaceutic agents including, but not limited to, antibiotics.

The invention is susceptible to various modifications and alternative means, and specific examples thereof have been shown by way of example in the drawings and are herein described in detail. It should be understood, however, that the invention is not to be limited to the particular devices or methods disclosed, but to the contrary; the invention is to cover all modifications, equivalents, and alternatives falling within the spirit and scope of the claims.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A closure device for clamping sutures, the closure device comprising:
an inner part having a distal end and a proximal end and having an opening formed therein extending from the distal end to the proximal end; and
an outer part having an aperture configured to receive the inner part, wherein the outer part and the inner part cooperate to clamp sutures drawn through the opening when the inner part is received in the aperture of the outer part.

2. The closure device of claim 1, the inner part further comprising an outer wall extending between the proximal end and the distal end, wherein the outer wall is configured to match an inner wall of the outer part.

3. The closure device of claim 2, wherein the inner wall and the outer wall are tapered.

4. The closure device of claim 2, wherein the outer wall includes first projections oriented towards the proximal end and wherein the inner wall includes second projections oriented towards a distal end of the outer part.

5. The closure device of claim 4, wherein the first projections and the second projections cooperate to enable insertion of the inner part into the aperture and resist removal of the inner part from the aperture of the outer part.

6. The closure device of claim 4, wherein the outer part comprises a material that is more rigid that a material of the inner part.

7. The closure device of claim 4, wherein the first projections are spaced on the outer wall and are placed between the proximal end and the distal end of the outer wall, wherein the second projections are spaced on the inner wall and are placed between the proximal end and the distal end of the inner wall to correspond to the placement of the first projections.

8. The closure device of claim 1, wherein the inner part is larger than the aperture such that insertion of the inner part into the aperture closes the opening to clamp the sutures, wherein the outer part exerts a compressive force on the inner part to close the opening on the sutures.

9. A closure device for holding sutures closing an opening in tissue, the closure device comprising:
an inner part having an opening formed therein, the opening extending from a distal end to a proximal end;
a first engagement mechanism formed on an outer surface of the inner part; and
an outer part having a second engagement mechanism formed on an inner surface, wherein the second engagement mechanism engages the first engagement mechanism to connect the inner part with the outer part.

10. The closure device of claim 9, wherein the outer surface includes an outer wall that extends between the distal end and the proximal end, wherein the outer wall is adjacent an inner wall of the outer part when the inner part is connected with the outer part.

11. The closure device of claim 9, wherein the first engagement mechanism includes first projections formed on the outer surface and wherein the second engagement mechanism includes second projections formed on the inner surface that engage with the first projections to secure the inner part to the outer part.

12. The closure device of claim 11, wherein the first projections extend toward the proximal end of the inner part and the second projections extend toward a distal end of the outer part.

13. The closure device of claim 11, wherein the inner part elastically deforms to close the opening on sutures drawn through the opening when the inner part is pressed into the outer part.

14. The closure device of claim 11, wherein the first projections are arranged circumferentially on the outer surface and wherein the second projections are arranged circumferentially on the inner surface.

15. The closure device of claim 9, wherein the first engagement mechanism and the second engagement mechanism allow the inner part to be pressed together with the outer part and resist removal of the inner part from the outer part.

16. The closure device of claim 9, wherein the inner part comprises a material that is less rigid that a material of the outer part.

17. The closure device of claim 9, wherein the outer part comprises a body defining an aperture for receiving the inner part, the body having a greater thickness at a distal end and a smaller thickness at the proximal end.

18. A method for closing an opening in tissue, the method comprising:
placing sutures around the opening in the tissue;
drawing the sutures through a closure device, the closure device including an inner part and an outer part;
tightening the sutures; and
pressing the inner part together with the outer part to clamp the sutures in the closure device and hold the opening in the tissue closed.

19. The method of claim 18, wherein drawing the sutures through a closure device further comprises drawing the sutures through an aperture of the outer part and through an opening of the inner part, wherein pressing the inner part together with the outer part closes the opening on the sutures.

20. The method of claim 19, wherein pressing the inner part together with the outer part further comprises engaging first projections formed on the inner part with second projections formed on the outer part, wherein the first projections and the second projections allow the inner part to be pressed into the outer part and resist separation of the inner part and the outer part.

21. A system for closing an opening in tissue, the system comprising:
a closure device configured to clamp sutures drawn through the closure device,
the closure device including a first part and a second part; and
a delivery device including:
a shaft having a proximal end and a distal end, the distal end having a recess shaped to receive the first part of the closure device, wherein distal movement of the delivery device engages the first part with the second part after sutures are drawn through the first part and the second part.

22. The system of claim 21, wherein the first part comprises an outer part and the second part comprises an inner part, the inner part received in an aperture of the outer part when the inner part is pressed into the outer part.

23. The system of claim 21, wherein the shaft comprises an opening for receiving sutures such that the sutures extend out of the proximal end of the shaft when deploying the closure device.
